(19) United States
(12) Patent Application Publication
(10) Pub. No.: US 2023/0222176 A1
(43) Pub. Date: Jul. 13, 2023

(54) DISEASE REPRESENTATION AND CLASSIFICATION WITH MACHINE LEARNING

(71) Applicant: X Development LLC, Mountain View, CA (US)

(72) Inventors: Garrett Raymond Honke, Mountain View, CA (US); Baihan Lin, New York, NY (US); Anupama Thubagere Jagadeesh, San Jose, CA (US)

(21) Appl. No.: 17/574,915

(22) Filed: Jan. 13, 2022

(51) Int. Cl. G06K 9/62; G01N 33/68; G01N 33/50

(52) U.S. Cl. CPC G06K 9/6256; G06K 9/623; G01N 33/6848; G01N 33/5038

(57) ABSTRACT

The invention features a computer-implemented biological data classification method executed by one or more processors and including receiving, by the one or more processors, a first biological data set comprising a first plurality of biological sample data collected from a set of patients; processing, by the one or more processors, the first biological data set using a first variational autoencoder (VAE) to generate a first trained VAE comprising a first latent space vector of the first biological data set comprising a plurality of values corresponding to each latent space dimension of the latent space vector, the latent space vector having lower dimensionality than the biological sample data set; receiving, by the one or more processors, a second biological data set comprising a second plurality of biological sample data collected from a patient, different from the set of patients; and generating, by the one or more processors, a latent space representation of the second biological data set based on a first latent space vector.

### FIELD OF THE DISCLOSURE

The disclosure relates to a machine learning system that generatively produces a disentangled representation of biological sample data and classifies biological sample data decoded from the disentangled representation.

### BACKGROUND

Medical entities, e.g., insurance company, doctor's office, hospital, urgent care, pharmacy, or testing facilities, etc., store and manage biological data for many patients across a large number of sampling events and testing modalities. As an example, a patient interacting with one or more medical entities for the length of their life generates biological data across multiple data dimensions. An interaction with a medical entity can generate biological sample data including a height, weight, blood pressure, a respiration rate, analyte presence, quantities, or volumes in a sample, such as a blood, serum, interstitial fluid, saliva, or urine sample, etc. Medical sampling data generated from these testing modes is generally categorized or classified based on existing diagnoses from a historical diagnosis register. The existing tables of diagnoses were generated before high-throughput sampling and broad-spectrum testing made high-dimensional information of a biological sample available. Additionally, sparsely populated biological data having unknown or intermittent time intervals between sampling events results in a sparse matrix of un-correlated data.

### SUMMARY

In general, the disclosure relates to a machine learning system that generatively produces a disentangled representation of multi-dimensional matrices of biological sample data. Training the machine learning system on biological sample data generates a latent representation of the training sample data including analyte and disease representations. The training sample data can include biological information collected from a set of patient samples such as analyte labels, analyte volumes, patient ID, or sampling mode.

The resulting latent representation clusters data processed through the representation and can be used to classify disease states from learned correlated variables in the data. Once the latent representation has been generated, the system receives subsequent sample data and processes the data in an unsupervised manner to generatively produce a disentangled representation of analyte and disease classifications in the subsequent sample data, such as predict absent analyte volumes, and identify new disease predictors and early warning biomarkers, independent of sample type, patient cohort, or collection interval. The model is generalizable across sample mediums, e.g., blood, serum, saliva, interstitial fluid, and characterizes disease states across cohorts of patients.

In general, in a first aspect, the invention features a computer-implemented biological data classification method executed by one or more processors and including receiving, by the one or more processors, a first biological data set comprising a first plurality of biological sample data collected from a set of patients; processing, by the one or more processors, the first biological data set using a first variational autoencoder (VAE) to generate a first trained VAE comprising a first latent space vector of the first biological data set comprising a plurality of values corresponding to each latent space dimension of the latent space vector, the latent space vector having lower dimensionality than the biological sample data set; receiving, by the one or more processors, a second biological data set comprising a second plurality of biological sample data collected from a patient, different from the set of patients; and generating, by the one or more processors, a latent space representation of the second biological data set based on a first latent space vector.

Embodiments may include one or more of the following features. The first VAE can be a βVAE. The first biological data set can include analyte volumes in collected biological samples. The collected biological samples can be blood, serum, saliva, plasma, interstitial fluid. The analyte volumes can include protein volumes, or metabolite volumes. The method can include corrupting the received data set using a corruption function. Prior to receiving, the method can include corrupting or ablating the first biological data set. The corruption function can be a salt and pepper function, a Gaussian function, or a masking function. A loss function of the first VAE can be a forward KL divergence model, or a reverse KL divergence model.

The method can include: receiving, by the one or more processors, a classification label data set including a plurality of classification label constructed from biological sample data labels; processing, by the one or more processors, the classification label data set using a second VAE to generate a second trained VAE including a second latent space vector of the classification label data set, the second latent space vector including a plurality of values corresponding to each latent space dimension of the classification label data, the latent representation having lower dimensionality than the classification label data set; communicating, by the one or more processors, the latent space representation of the second biological data set to the second VAE; and classifying, by the one or more processors, the latent space representation of the second biological data set based on a first latent space vector. The classifying can include generating a disease prediction based on the second biological data set. The method can further include: receiving, by the one or more processors, a set of classifications including a plurality of sample classifications; processing, by the one or more processors, the set of classifications using the second VAE to generate a classification representation of the set of classifications, the second latent representation having lower dimensionality than the set of classifications; communicating, by the one or more processors, the classification representation to the first VAE; and generating, by the one or more processors, a predicted biological data set based on the classification representation.

In general, in a second aspect, the invention features a system including at least one processor; a storage device coupled to the at least one processor having instructions stored thereon which, when executed by the at least one processor, causes the at least one processor to perform operations including receiving, by the one or more processors, a first biological data set comprising a first plurality of biological sample data collected from a set of patients; processing, by the one or more processors, the first biological data set using a first variational autoencoder (VAE) to generate a first trained VAE comprising a first latent space vector of the first biological data set comprising a plurality of values corresponding to each latent space dimension of the latent space vector, the latent space vector having lower dimensionality than the biological sample data set; receiving, by the one or more processors, a second biological data set comprising a second plurality of biological sample data collected from a patient, different from the set of patients; and generating, by the one or more processors, a latent space representation of the second biological data set based on a first latent space vector.

Embodiments may include one or more of the following features. The data store can further include instructions stored thereon including a second VAE. The data store can further include instructions stored thereon which, when executed by the at least one processor, causes the at least one processor to perform operations including: receiving, by the one or more processors, a classification label data set including a plurality of classification label constructed from biological sample data labels; processing, by the one or more processors, the classification label data set using the second VAE to generate a second trained VAE including a second latent space vector of the classification label data set, the second latent space vector including a plurality of values corresponding to each latent space dimension of the classification label data, the latent representation having lower dimensionality than the classification label data set; communicating, by the one or more processors, the latent space representation of the second biological data set to the second VAE; and classifying, by the one or more processors, the latent space representation of the second biological data set based on a first latent space vector.

The data store can further include instructions stored thereon which, when executed by the at least one processor, causes the at least one processor to perform operations including: receiving, by the one or more processors, a set of classifications including a plurality of sample classifications; processing, by the one or more processors, the set of classifications using the second VAE to generate a classification representation of the set of classifications, the second latent representation having lower dimensionality than the set of classifications; communicating, by the one or more processors, the classification representation to the first VAE; generating, by the one or more processors, a predicted biological data set based on the classification representation.

In general, in a third aspect, the invention features a non-transitory computer readable storage medium storing instructions that when executed by at least one processor, cause the at least one processor to perform operations including receiving, by the one or more processors, a first biological data set comprising a first plurality of biological sample data collected from a set of patients; processing, by the one or more processors, the first biological data set using a first variational autoencoder (VAE) to generate a first trained VAE comprising a first latent space vector of the first biological data set comprising a plurality of values corresponding to each latent space dimension of the latent space vector, the latent space vector having lower dimensionality than the biological sample data set; receiving, by the one or more processors, a second biological data set comprising a second plurality of biological sample data collected from a patient, different from the set of patients; and generating, by the one or more processors, a latent space representation of the second biological data set based on a first latent space vector.

Among other advantages, a trained autoencoder model has the potential to determine heretofore unknown analyte-to-analyte volume correspondences. The difference between predictions and real-world values can help practitioners identify potentially important anomalies in patient data. Analyte correspondences can also be used to discover early warning biomarkers and general disease predictors across large cohorts of patients.

A trained autoencoder model creates data-driven disease taxonomies based on the learned disease representations in the latent vector space. The new disease taxonomies are based on the learned correlations between unlabeled analyte volumes which improve disease classification accuracies above observation and symptom-driven taxonomies and diagnoses.

The trained model can be used to diagnose disease states earlier and faster than traditional analyte collection modes. The latent vector space can span multiple collection modes (e.g., swab sample collection, blood collection, stool collection, urine collection) and facilitate data imputation in un-sampled dimensions, reducing the testing burden on patients while increasing the data collection rate. Improved disease classification accuracy increases the efficacy of follow-on treatments based on the model diagnosis.

The trained model can create longitudinal predictions along one or more latent vector space dimensions, enabling forward- and backward prediction of analyte volumes. Longitudinal prediction of analyte volumes across multiple dimensions enables increased testing specificity and disease prediction accuracy.

The trained autoencoder-driven clustering provides additional disease representations not determined through high-level observational human disease diagnoses from a healthcare providers. The data clustering differentiates between disease representations in an unsupervised manner to produce diagnoses between unique disease states having significantly overlapping symptomology.

Other advantages will be apparent from the description, the drawings, and the claims.

### BRIEF DESCRIPTION OF THE DRAWINGS

FIG. 1 is a schematic representation of a system for producing a clustered representation of biological sample data.

FIGS. 2 and 3 are schematic illustrations of processing a patient data set through a VAE processing system.

FIG. 4 is schematic illustration of a classifying a patient data set through a VAE classification system.

FIG. 5 is a work flow diagram of a process for processing a patient data set through a VAE processing system.

FIG. 6 is a work flow diagram of a process for classifying a patient data set through a VAE classification system.

FIG. 7 is a schematic representation of a computing device.

In the figures, like symbols indicate like elements.

### DETAILED DESCRIPTION

Generally, testing results from biological samples collected from a set of patients include information related to a presence, quantity, or volumes of one or more analytes. Fluid samples, including blood, serum, interstitial fluid, saliva, or urine, can produce thousands of analyte volumes in a single sampling. The information can be used to determine one or more disease states correlated with the presence or quantity of one or more analytes. For example, blood glucose levels higher than a pre-determined level within a biological sample can indicate the presence of a diabetic disease state. The methods and systems of this disclosure receive unlabeled biological sample data from a set of patients, e.g., biological sample data which has had patient and analyte identifying label information removed, and process the data through a variational autoencoder (VAE).

Processing the collected biological sample data from a set of patients through the

VAE trains the program to reduce the dimensionality of the sample data to eigenvalues of the latent variable space which represents the collected biological sample data as a vector of latent variables. Each variable in the lower-dimensional representation learns a particular latent variable corresponding to a dimension of the biological sample data.

Using the autoencoder to encode unlabeled biological data and feeding the resulting lower-dimensional encoding to an unsupervised clustering algorithm (e.g., UMAP, HDBSCAN, reciprocal agglomerative clustering) will produce groupings (clusters) of biological sample data based on the learned latent vector representation. Each cluster of biological data contains information that characterizes the corresponding biological feature. Correlations between clusters can be used, e.g., for determining a previously undiagnosed disease state.

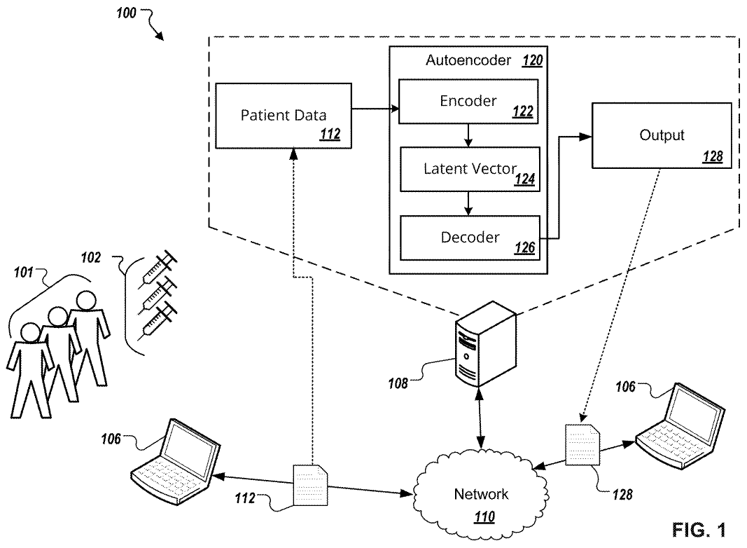

FIG. 1 is a diagram that illustrates an example system 100 for generatively produces a disentangled representation of multi-dimensional matrices of biological sample data using a variational autoencoder (VAE). The system 100 includes a data clustering system (DCS) 108 in communication with a plurality of healthcare computing devices 106 over a network 110. The network 110 can include public and/or private networks and can include the Internet.

The DCS 108 can include a system of one or more computers. In general, the DCS 108 is configured to perform one or more types of machine learning processes on a combination of biological sample data to assign unlabeled biological data to a previously generated cluster based on a trained latent vector representation of patient biological data. The sorting and/or grouping can be performed automatically without any user input. The user may also be enabled, e.g., provided with a user interface, to interact with the sorting and/or grouping.

The DCS 108 obtains patient data 112 over a period of time (e.g., a period of days, weeks, or months) including over multiple collection events. The biological sample data 112 includes measurements of various biological parameters received from the computing devices 106, e.g., entered by a technician at a testing site or entered by a high-throughput sample screening system. The biological sample data 112 can include results from biological tests 102 performed on a set of patients 101.

In some implementations, data access rules executed by the DCS 108 permit the DCS 108 to obtain biological sample data 112 without third-party human interaction with the data on the DCS 108, thereby, protecting patient privacy. The DCS 108 can further protect each patient's privacy by the DCS 108 assigning anonymized patient identifiers to each set of patients 101 whose data is obtained. The DCS 108 can use the anonymized patient identifiers to correlate data to specific patients while protecting personal information. For example, the system can remove personally identifiable information and assign a unique patient identifier to each unique patient. In some examples, the patient identifiers may be non-reversible to protect each patient's identity. In some examples, the system can perform a cryptographic hash function on particular aspects of each patient's identity, e.g., the system can hash a combination of the patient's name, address, and date of birth to obtain a unique patient identifier.

In various implementations, DCS 108 can perform some or all of the operations related to clustering and/or classifying biological sample data 112. For example, DCS 108 can include an autoencoder 120 and a decoder 126. The autoencoder 120 and decoder 126 can each be provided as one or more computer executable software modules or hardware modules. That is, some or all of the functions of autoencoder 120 and decoder 126 can be provided as a block of code, which upon execution by a processor, causes the processor to perform functions described below. Some or all of the functions of autoencoder 120 and decoder 126 can be implemented in electronic circuitry, e.g., as field programmable gate array (FPGA) or an application specific integrated circuit (ASIC).

In operation, DCS 108 receives biological sample data 112 from set of patients 101. The biological sample data 112 is provided to the DCS 108 over the network 110, or from one or more computing devices 106 connected to the DCS 108. More specifically, biological sample data 112 can include multiple streams or “channels” of different types of patient biological data, e.g., blood sample data, or interstitial fluid sample data. In some implementations, the biological sample data 112 includes time-series patient biological data, e.g., biological data collected in more than one biological test 102 over a period of time.

The DCS 108 may accumulate biological sample data 112 over the course of several days or weeks before analyzing the biological sample data 112 using the autoencoder 120, e.g., to ensure sufficient data is available to generate a latent vector representation for the set of patients 101. In some implementations, the DCS 108 may accumulate biological sample data 112 until a quantity of data is achieved, e.g., until data from a specified number of patients is collected. The number of patients in the set of patients 101 can include thousands, or millions, of patients to achieve a required training level of the DCS 108.

The DCS 108 then applies a series of one or more machine learning algorithms to the biological sample data 112 to generate a low-rank representation of the biological data. In some implementations, the machine learning algorithms can be a neural network. A neural network is a machine learning model that includes one or more input layers, one or more output layers, and one or more hidden layers that each apply a transformation to a received input to generate an output. In some implementations, the neural network may be an autoencoder 120. In some implementations, the neural network may be a variational autoencoder (VAE). In some implementations, the neural network may be a β-variational autoencoder (β-VAE).

The DCS 108 includes a VAE autoencoder 120 which stores and executes one or more variational autoencoder algorithms and processes the biological sample data 112 to generate a biological data latent variable representation, e.g., latent vector 124.

The DCS 108 applies the biological sample data 112 as input to the autoencoder 120. The autoencoder 120 includes modules which execute a principle component analysis algorithm to generate a latent vector representation of the biological sample data 112. An autoencoder 120 is a neural network designed to construct a latent vector 124 of an input in an unsupervised way. The autoencoder 120 includes an encoder 122 which generates a latent vector 124 by reducing the dimensionality of the input biological sample data 112, thereby compressing the biological sample data 112 provided to the DCS 108.

The latent vector 124 includes one or more hidden variables with which the autoencoder 120 has been trained to represent components in the biological sample data 112. The hidden variables within the latent vector 124 can have one or more scores representing components of the biological sample data 112 on which the autoencoder 120 has been trained. In general, the dimensionality of the input biological sample data 112 can be larger than the dimensionality of the latent vector 124.

The encoder 122 can have one or more input layers to receive the biological sample data 112. In general, the encoder 122 can process received biological sample data 112, individually or in parallel. In general, the encoder 122 can process the data contained in biological sample data 112 or it can apply one or more modifying functions. In some implementations, the encoder 122 can duplicate at least a portion of the biological sample data 112 before applying a modifying function, e.g., a domain transformation function, or a corruption function.

In some implementations, the encoder 122 can apply a corruption function to partially sample the biological sample data 112. The corruption function can include using a sampling distribution to partially sample the biological sample data 112. The sampling distribution can include a Gaussian distribution, masking distribution, salt-and-pepper distribution, or other distributions.

The autoencoder 120 includes a decoder 126 to construct an output 128 from the latent vector 124. After the encoder 122 has determined the latent vector 124 of the received biological sample data 112, In general, the decoder 126 can be one or more hidden layers within the autoencoder 120 neural network. The autoencoder 120 can execute the decoder 126 to attempt to construct an output 128 that is similar to the input biological sample data 112. Once the autoencoder 120 has constructed an output 128, the autoencoder 120 can then use a loss function to determine the difference, e.g., loss, between the output 128 and the input biological sample data 112. For example, the loss function an autoencoder 120 uses can be a mean squared error (MSE) function or MSE plus a K-L divergence loss function. The K-L divergence loss function can be a forward K-L divergence loss function or a reverse K-L divergence loss function.

After determining the loss between an output 128 and the input biological sample data 112, the autoencoder 120 can update one or more variables within the latent vector 124 to attempt to reconstruct the output 128 to achieve a lower loss. If a lower loss is achieved, the autoencoder will continue to process biological sample data 112 with the updated latent vector 124. The updated latent vector 124 can then be used to encode and decode one or more biological sample data 112 before calculating a new loss. In this manner, the autoencoder 120 can be trained to reconstruct input biological sample data 112 from the latent vector 124 with the lowest loss. The autoencoder 120 can be considered trained once the decoder 126 determines that the loss is below a loss threshold, e.g., at a loss minimum determined by the decoder 126 or stored by the autoencoder 120.

A large set of biological sample data 112 from a large set of patients 101 can train the autoencoder 120 to achieve a minimum loss below the loss threshold. For example, the DCS 108 can receive biological sample data 112 from thousands, or millions of patients before the loss threshold is achieved and the autoencoder 120 is considered trained.

The decoder 126 generates predicted, e.g., imputed, biological data by projecting the latent vector 124 into a reconstructs the biological sample data 112 into output 128 which includes predicted biological data. The decoder 126 constructs the predicted biological data into the biological sample data 112 where there are empty elements along the dimensions of the biological sample data 112. The output 128 includes predicted biological data along one or more dimensions.

Once sufficient biological sample data 112 has been collected for a particular set of patients 101, the DCS 108 can update the analysis of the set of patients 101 data at regular intervals (e.g., daily, weekly, or month) by incorporating the data received over the time interval with the set of patients 101 past biological data. The collected biological sample data 112 can be correlated with a patient, a patient identifier, or the collected patient data in the patient data set 204 can be anonymized by the DCS 108, e.g., patient data decorrelated with identifying information. Alternatively, the biological sample data 112 is anonymized before the DCS 108 receives the biological sample data 112. The DCS 108 stores the output 128 for access from one or more devices, e.g., healthcare computing devices 106, over the network 110. Alternatively, the DCS 108 can send the output 128 over the network 110 to the healthcare computing devices 106, for access by and/or display to individual users or clinicians.

The output 128 contains the biological sample data 112 and the predicted biological data. From the output 128, information relating to interpretable insights into the health status of a user, or the collective health status of the set of patients 101, can be determined by further processing or access by a healthcare entity, such as an insurance company, doctor's office, hospital, urgent care, or pharmacy. As an example, a doctor may determine that a patient of the set of patients 101 has a previously undiagnosed disease based on the output 128. As a second example, a hospital may determine that one or more patients of the set of patients 101 requires additional testing, e.g., testing frequency or testing modes, based on interpreting the output 128.

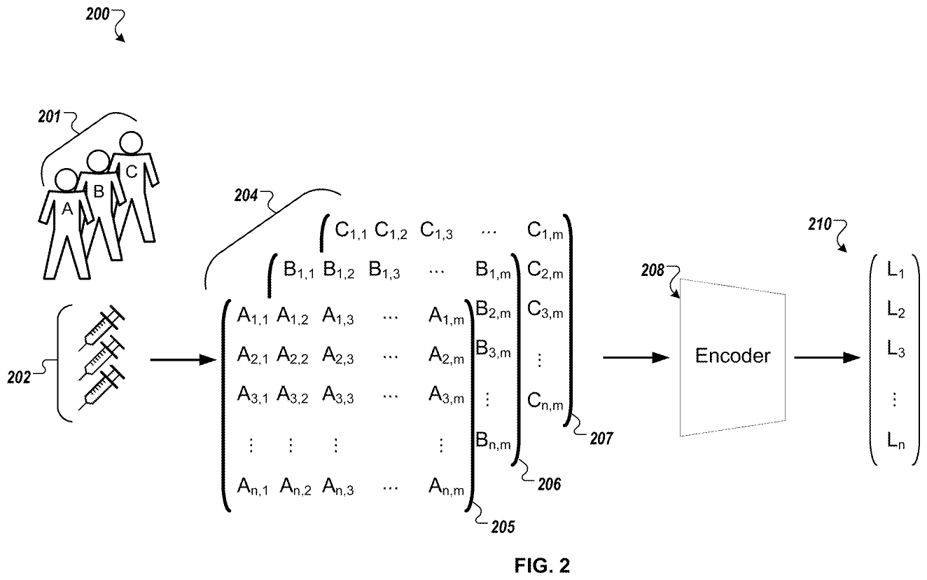

The biological sample data 112 is constructed from biological data collected from a set of patients 101 and processed by the encoder 122 of the autoencoder 120. Referring to FIG. 2, a schematic illustration of processing a patient data set 204 generated from biological tests 202 on a set of patients 201 through a VAE processing system 200 is shown. The VAE processing system 200 is representative of the encoding portion of the autoencoder 120 of FIG. 1. A set of patients 201 generates biological sample data when biological tests 202 are performed. The biological tests 202 can include any biological test described herein. In some implementations, the patient data set 204 is constructed from biological data related to a single patient. In alternative implementations, the patient data set 204 is constructed from patient data related to more than one patient, such as the set of patients 201.

The patient data set 204 is constructed from a number of biological data samples, e.g., biological data samples 205, 206, and 207, which each correspond to one patient of the set of patients 201, e.g., biological data sample 205 206 and 207 correspond to patients A, B, and C, respectively. The data included in the biological data samples 205, 206, and 207 corresponds to the type or mode of the biological tests 202. For example, a blood serum test may determine the presence or quantity of one set of analytes, while an interstitial fluid test may determine the presence or quantity of a second set of analytes, having one or more overlapping analytes with the first set.

The dimensionality of biological data samples 205, 206, and 207, constructing patient data set 204, corresponds to the number of dimensions of the collected biological data from the set of patients 201. A dimension of the patient data set 204 can be time, e.g., the patient data set 204 can be generated over a time period. The time period over which the biological data is collected can be different for each example, or it can be the same. Biological data sample 205 is shown having a set of dimensions represented as an n by m matrix relating to patient A. Biological data samples 206 and 207 are shown with similar dimensions, relating to patients B and C respectively.

In some implementations, the patient data set 204 includes the presence or quantity of a biomarker present in a sample collected from a patient at a time point or series of time points. Examples of biomarkers can include red blood cells, white blood cells, platelets, sodium, potassium, magnesium, nitrogen, carbon dioxide, oxygen, glucose, Vitamin A, Vitamin D, Vitamin B1 (thiamine), Vitamin B12, folate, calcium, Vitamin E, Vitamin K, zinc, copper, Vitamin B6, Vitamin C, homocysteine, iron, hemoglobin, hematocrit, insulin, melanin, hormones, testosterone, estrogen, cortisol, thyroxine, triiodothyronine, human growth hormone, insulin-like growth factors, thyroid stimulating hormone (TSH), carotenoids, cytokines, interleukins, chlorides, cholesterols, lipoproteins, triglycerides, c-peptide, creatinine, creatine, creatine kinase, urea, ketones, peptides, proteins, albumin, bilirubin, myoglobin, ESR, CRP, IL6, immunoglobins, resistin, ferritin, transferrin, antigens, troponins, gamma-glutamyltransferase (GGT), lactate dehydrogenase (LD), alanine aminotransferase, alkaline phosphatase, and aspartate aminotransferase.

As shown in FIG. 2 and described above, the VAE processing system 200 includes an autoencoder 220. An autoencoder 220 is a neural network designed to construct a latent vector 210 of an input in an unsupervised way. By using an encoder 208 to compress the patient data set 204 within a received Patient data set 204 into a latent vector 210 it can greatly reduce the dimensionality of the input patient data set 204. The latent vector 210 can include one or more hidden variables with which the autoencoder 220 has been trained to represent components in input patient data set 204. The hidden variables within the latent vector 210 can have one or more scores representing components of the patient data set 204 on which the autoencoder 220 has been trained. In general, the dimensionality of the input patient data set 204 can be larger than the dimensionality of the latent vector 210.

In general, the encoder 208 can have one or more input layers to receive one or more patient data sets 204. The encoder 208 can process the one or more patient data sets 204 individually or in parallel. In some implementations, the encoder 208 processes a portion of the one or more patient data set 204 to determine a latent vector 210. The encoder 208 can process the unmodified data contained in the patient data set 204 or it can apply one or more modifying functions. In some implementations, the encoder 208 can duplicate one or more patient data sets 204 before applying a modifying function, e.g., a corruption function.

In some implementations, the encoding engine can apply a corruption function to partially sample the patient data set 204. The corruption function can include using a sampling distribution to partially sample the patient data set 204. The sampling distribution can include a Gaussian distribution, masking distribution, salt-and-pepper distribution, or other distributions.

The encoder 208 generates the trained latent vector 210 by processing the patient data set 204 and outputting a representation of the patient data set 204 having eigenvalues of the latent variable space representing the patient data set 204.

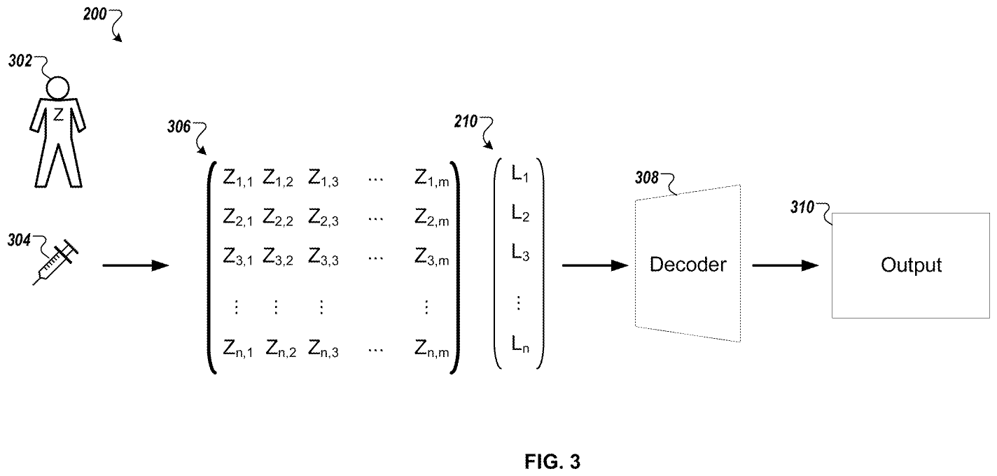

The VAE processing system 200 uses the trained latent vector 210 to project data into an output having missing data imputed or additional synthetic data generated within the a new patient data set. FIG. 3 is a schematic illustration of processing a patient data set 304 from a single patient 301 including a biological data sample 306 through the decoder 308 of the VAE processing system 200.

The VAE processing system 200 receives a patient data set 304 including a biological data sample 306 from a single patient 301, Z. The biological data sample 306 is shown as an n by m matrix having the same or substantially similar data dimensions as the patient data sets 204 on which the VAE processing system 200 was trained.

The VAE processing system 200 processes the biological data sample 306 through a decoder 308. As described above, the decoder 308 can be one or more hidden layers within the VAE processing system 200 neural network. The VAE processing system 200 executes the decoder 308 and constructs output 310.

The output 310 generated by a trained VAE processing system 200 based on received patient data set 304 from a single patient 301 is a reconstructed patient data set having imputed data between the biological data sample 306. The decoder 308 projects the patient data set 304 through the latent vector 210 which expands the vector into a complete representation of the biological data sample 306 including the imputed data.

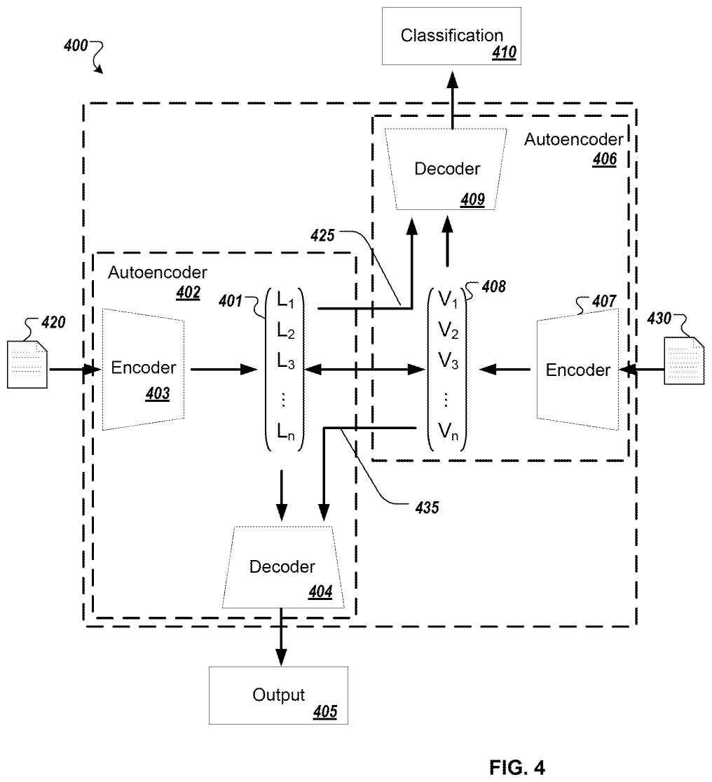

In some implementations, the autoencoder 120 of FIG. 1 can include more than one autoencoder system, e.g., two autoencoders. In such implementations, the autoencoder systems can be trained to map classification labels provided to the VAE processing system 200 to the latent vector 210 representations. FIG. 4 is a schematic illustration of a classification system 400 having a reconstruction autoencoder 402 which receives biological sample data 420 and a classification autoencoder 406 which receives classification label data 430. The classification system 400 generates a classification 410 and/or an output 405 based on the input to the reconstruction autoencoder 402 and the classification autoencoder 406.

The classification system 400 can be trained by receiving biological sample data 420 and processing the biological sample data 420 through the layers of the encoder 403 and creating a trained reconstruction latent vector 401. The reconstruction autoencoder 402 includes a decoder 404 for projecting received biological sample data 420 into an imputed output 405.

The classification autoencoder 406 is trained using the trained reconstruction latent vector 401 of the reconstruction autoencoder 402. The classification autoencoder 406 processes the classification label data 430 through the layers of encoder 407 into a classification latent vector 408 in which the elements are eigenvalues of the latent variable space representing the classification labels provided. The classification label data 430 includes data labels representing categorical labels related to biological data. Such classification categories can include age, weight, height, analyte labels, quantities, concentrations, amounts, diagnoses, disease states, or other biologically appropriate label.

When the reconstruction autoencoder 402 and classification autoencoder 406 of the classification system 400 are fully trained, the classification system 400 can receive biological sample data 420 into the reconstruction autoencoder 402 and reduce the data to a vector of values along the eigenvector dimensions of the reconstruction latent vector 401. Following path 425, the biological sample data 420 representation is provided to decoder 409 of the classification autoencoder 406. The representation is projected out of the decoder 409 into classification 410 based on the representation. In this manner, biological sample data 420 can be classified, e.g., labeled, based on the trained vectors 401 and 408.

For example, biological sample data 420 which has been anonymized and having all data labels removed can be processed through encoder 403 and decoder 409 to provide estimated data labels, e.g., classification 410, based on the trained classification system 400.

Alternatively, the classification system 400 processes classification label data 430 through the encoder 407 to reduce the label data to a vector of values along the eigenvector dimensions of the classification latent vector 408. Following path 435, the representation is projected out of the decoder 404 into output 405 based on the representation. In this manner, classification label data 430 can be projected into imputed biological sample data, e.g., simulated biological sample data, based on the trained vectors 401 and 408.

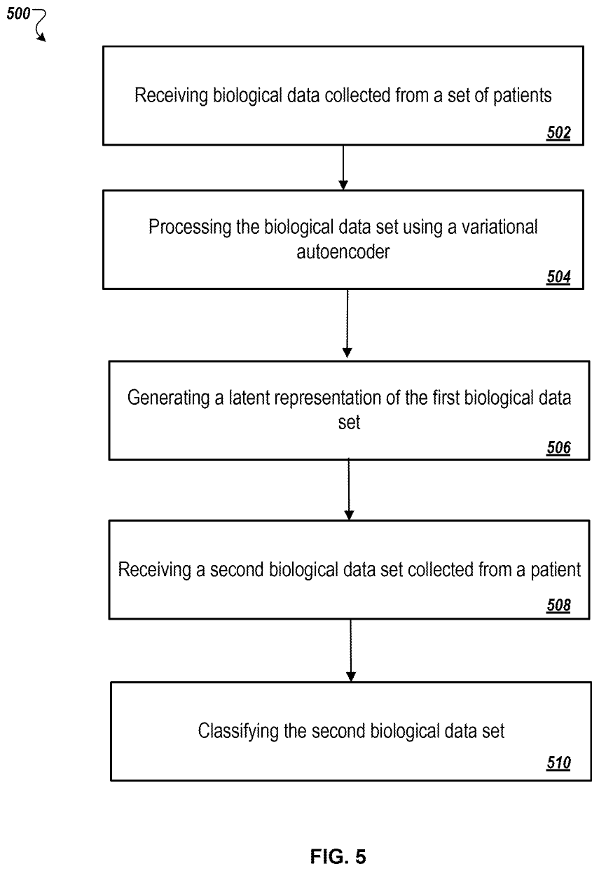

FIG. 5 is a flow-chart diagram of the individual steps for generatively producing a disentangled representation of multi-dimensional matrices of biological sample data using a variational autoencoder (VAE) (500).

A DCS 108 receives biological sample data 112 from a set of patients 101 (502). The biological sample data 112 can be received over a network 110, from a computing device 106 attached to the network 110, or a computing device 106 connected to the DCS 108. The biological sample data 112 includes a matrix of biological data collected in one or more biological tests 102 from the set of patients 101. The DCS 108 accumulates biological sample data 112 until a quantity of data is achieved, e.g., until data from a specified number of patients is collected.

The DCS 108 applies the biological sample data 112 to the autoencoder 120 as input. The autoencoder 120 processes the biological sample data 112 using an encoder 122, such as a variational autoencoder (504). The autoencoder 120 can apply the biological sample data 112 to one or more layers of the encoder 122 which receives the biological sample data 112. In general, the encoder 122 can apply one or more modifying functions. In some implementations, the encoder 122 can duplicate at least a portion of the biological sample data 112 before applying a modifying function, e.g., a domain transformation function, or a corruption function. For example, the corruption function can be a salt and pepper function, a Gaussian function, or a masking function.

The autoencoder 120 generates a latent vector 124 representation of the biological sample data 112 by reducing the dimensionality of the input biological sample data 112 (506). The latent vector 124 is processed through a decoder 126 to create an output 128. The output 128 is compared to the biological sample data 112 and a loss determined between the output 128 and the biological sample data 112. The autoencoder 120 updates one or more variables within the latent vector 124 to attempt to reconstruct the output 128 to achieve a lower loss. The autoencoder 120 is trained once the decoder 126 determines that the loss is below a loss threshold, e.g., at a minimum.

The DCS 108 receives a second set biological sample data 112 from a patient (508). The patient from which the second set biological sample data 112 is received can be a patient of the set of patients 101, or the patient can be independent from the set of patients 101.

The DCS 108 processes the biological sample data 112 from a patient through the autoencoder 120 to reconstruct the biological sample data 112 and generate a reconstructed biological sample data (510). The autoencoder 120 processes the biological sample data 112 from the patient through the encoder 122 to generate a latent space vector. The representation of the biological sample data 112. The autoencoder 120 processes the latent space representation through the decoder 126 to generated reconstructed biological sample data. The reconstructed biological sample data includes the biological sample data 112 received by the DCS 108 and data imputed by the autoencoder 120 to fill missing elements in the biological sample data 112 matrix.

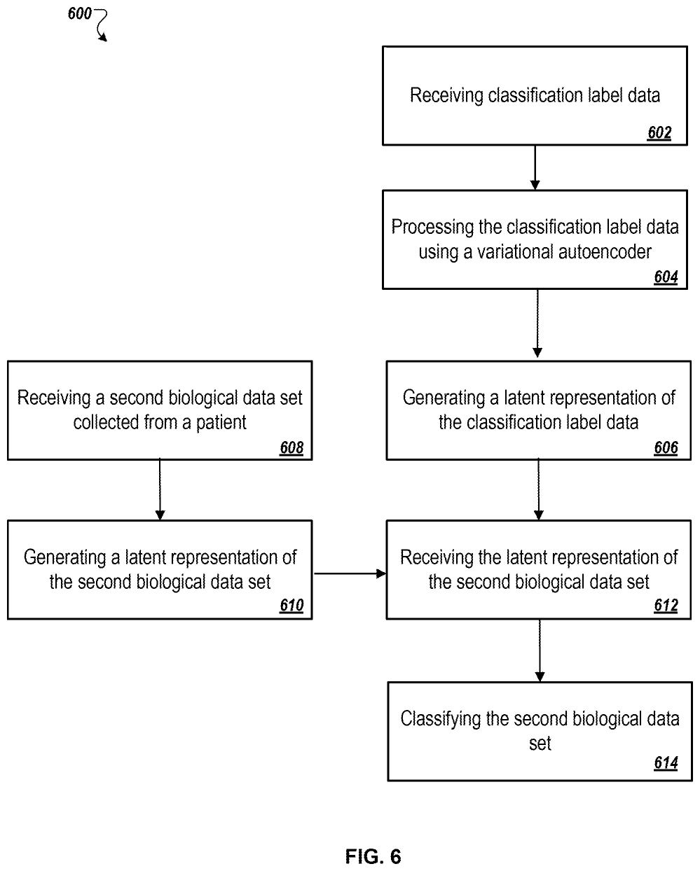

The trained autoencoder 120 having a trained latent vector 124 can be used in combination with a classification autoencoder 406 which learns a classification latent vector 408 based on the trained latent vector 124. FIG. 6 is a flow-chart diagram of the individual steps for generatively producing a classification of reconstructed biological sample data using a variational autoencoder (VAE) (600).

In some implementations, the DCS 108 includes a classification system 400 for classifying received biological sample data 112 based on a trained reconstruction latent vector 401 and a trained classification latent vector 408. A DCS 108 including a classification system 400 receives classification label data 430 corresponding to categorical labels related to biological data (602). The classification label data 430 can be received over a network 110, from a computing device 106 attached to the network 110, or a computing device 106 connected to the DCS 108. The classification label data 430 includes a matrix of biological data collected in one or more biological tests 102 from the set of patients 101. The DCS 108 accumulates classification label data 430 until a quantity of data is achieved, e.g., until data from a specified number of patients is collected.

The DCS 108 applies the classification label data 430 to the classification autoencoder 406 as input. The classification autoencoder 406 processes the classification label data 430 using an encoder 403, such as a variational autoencoder (604). The classification autoencoder 406 can apply the classification label data 430 to one or more layers of the encoder 403 which receives the classification label data 430. In general, the encoder 403 can apply one or more modifying functions.

The classification autoencoder 406 generates a classification latent vector 408 representation of the classification label data 430 by reducing the dimensionality of the input classification label data 430 (606). The classification latent vector 408 is processed through a decoder 409 to create a classification 410. The classification 410 is compared to the classification label data 430 and a loss determined between the classification 410 and the classification label data 430. The classification autoencoder 406 updates one or more variables within the classification latent vector 408 to attempt to reconstruct the classification 410 to achieve a lower loss. The classification autoencoder 406 is trained once the decoder 409 determines that the loss is below a loss threshold, e.g., at a minimum.

As described above, the DCS 108 receives a second set biological sample data 420 from a patient (608). The DCS 108 processes the biological sample data 420 from a patient through the reconstruction autoencoder 402 to generate a latent space representation of the biological sample data 420 (610). The latent space representation includes weights along each of the trained reconstruction latent vector 401 dimensions representing the biological sample data 420 in the reduced latent space.

The classification autoencoder 406 receives the latent space representation from the reconstruction autoencoder 402 (612). The classification autoencoder 406 then processes the latent space representation through the decoder 409 using the classification latent vector 408 to reconstruct the representation into a classification 410 (614). The classification 410 includes labels provided to the classification autoencoder 406 in the classification label data 430 applied to the latent space representation of the biological sample data 420.

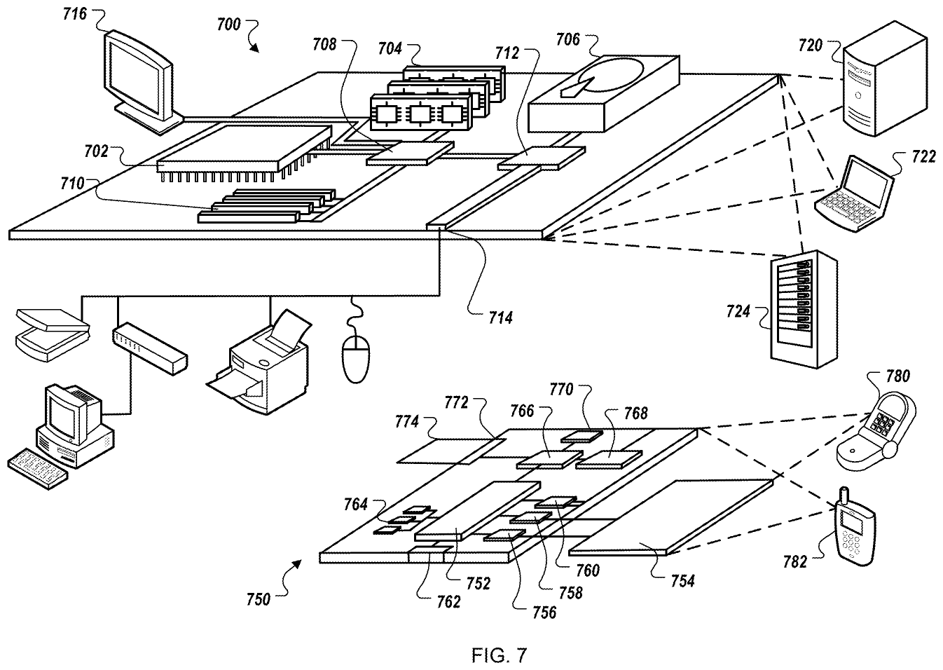

As noted previously, the systems and methods disclosed above utilize data processing apparatus to generatively producing a disentangled representation of multi-dimensional matrices of, or a classification of, biological sample data using one or more variational autoencoders (VAE) as described herein. FIG. 7 shows an example of a computing device 700 and a mobile computing device 750 that can be used as data processing apparatuses to implement the techniques described here. The computing device 700 is intended to represent various forms of digital computers, such as laptops, desktops, workstations, personal digital assistants, servers, blade servers, mainframes, and other appropriate computers. The mobile computing device 750 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 700 includes a processor 702, a memory 704, a storage device 706, a high-speed interface 708 connecting to the memory 704 and multiple high-speed expansion ports 710, and a low-speed interface 712 connecting to a low-speed expansion port 714 and the storage device 706. Each of the processor 702, the memory 704, the storage device 706, the high-speed interface 708, the high-speed expansion ports 710, and the low-speed interface 712, are interconnected using various busses, and may be mounted on a common motherboard or in other manners as appropriate. The processor 702 can process instructions for execution within the computing device 700, including instructions stored in the memory 704 or on the storage device 706 to display graphical information for a GUI on an external input/output device, such as a display 716 coupled to the high-speed interface 708. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 704 stores information within the computing device 700. In some implementations, the memory 704 is a volatile memory unit or units. In some implementations, the memory 704 is a non-volatile memory unit or units. The memory 704 may also be another form of computer-readable medium, such as a magnetic or optical disk.

The storage device 706 is capable of providing mass storage for the computing device 700. In some implementations, the storage device 706 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 702), perform one or more methods, such as those described above. The instructions can also be stored by one or more storage devices such as computer- or machine-readable mediums (for example, the memory 704, the storage device 706, or memory on the processor 702).

The high-speed interface 708 manages bandwidth-intensive operations for the computing device 700, while the low-speed interface 712 manages lower bandwidth-intensive operations. Such allocation of functions is an example only. In some implementations, the high-speed interface 708 is coupled to the memory 704, the display 716 (e.g., through a graphics processor or accelerator), and to the high-speed expansion ports 710, which may accept various expansion cards (not shown). In the implementation, the low-speed interface 712 is coupled to the storage device 706 and the low-speed expansion port 714. The low-speed expansion port 714, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 700 may be implemented in a number of different forms, as shown in the figure. For example, it may be implemented as a standard server 720, or multiple times in a group of such servers. In addition, it may be implemented in a personal computer such as a laptop computer 722. It may also be implemented as part of a rack server system 724. Alternatively, components from the computing device 700 may be combined with other components in a mobile device (not shown), such as a mobile computing device 750. Each of such devices may contain one or more of the computing device 700 and the mobile computing device 750, and an entire system may be made up of multiple computing devices communicating with each other.

The mobile computing device 750 includes a processor 752, a memory 764, an input/output device such as a display 754, a communication interface 766, and a transceiver 768, among other components. The mobile computing device 750 may also be provided with a storage device, such as a micro-drive or other device, to provide additional storage. Each of the processor 752, the memory 764, the display 754, the communication interface 766, and the transceiver 768, are interconnected using various buses, and several of the components may be mounted on a common motherboard or in other manners as appropriate.

The processor 752 can execute instructions within the mobile computing device 750, including instructions stored in the memory 764. The processor 752 may be implemented as a chipset of chips that include separate and multiple analog and digital processors. The processor 752 may provide, for example, for coordination of the other components of the mobile computing device 750, such as control of user interfaces, applications run by the mobile computing device 750, and wireless communication by the mobile computing device 750.

The processor 752 may communicate with a user through a control interface 758 and a display interface 756 coupled to the display 754. The display 754 may be, for example, a TFT (Thin-Film-Transistor Liquid Crystal Display) display or an OLED (Organic Light Emitting Diode) display, or other appropriate display technology. The display interface 756 may comprise appropriate circuitry for driving the display 754 to present graphical and other information to a user. The control interface 758 may receive commands from a user and convert them for submission to the processor 752. In addition, an external interface 762 may provide communication with the processor 752, so as to enable near area communication of the mobile computing device 750 with other devices. The external interface 762 may provide, for example, for wired communication in some implementations, or for wireless communication in other implementations, and multiple interfaces may also be used.

The memory 764 stores information within the mobile computing device 750. The memory 764 can be implemented as one or more of a computer-readable medium or media, a volatile memory unit or units, or a non-volatile memory unit or units. An expansion memory 774 may also be provided and connected to the mobile computing device 750 through an expansion interface 772, which may include, for example, a SIMM (Single In Line Memory Module) card interface. The expansion memory 774 may provide extra storage space for the mobile computing device 750, or may also store applications or other information for the mobile computing device 750. Specifically, the expansion memory 774 may include instructions to carry out or supplement the processes described above, and may include secure information also. Thus, for example, the expansion memory 774 may be provide as a security module for the mobile computing device 750, and may be programmed with instructions that permit secure use of the mobile computing device 750. In addition, secure applications may be provided via the SIMM cards, along with additional information, such as placing identifying information on the SIMM card in a non-hackable manner

The memory may include, for example, flash memory and/or NVRAM memory (non-volatile random access memory), as discussed below. In some implementations, instructions are stored in an information carrier. The instructions, when executed by one or more processing devices (for example, processor 752), perform one or more methods, such as those described above. The instructions can also be stored by one or more storage devices, such as one or more computer- or machine-readable mediums (for example, the memory 764, the expansion memory 774, or memory on the processor 752). In some implementations, the instructions can be received in a propagated signal, for example, over the transceiver 768 or the external interface 762.

The mobile computing device 750 may communicate wirelessly through the communication interface 766, which may include digital signal processing circuitry where necessary. The communication interface 766 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 768 using a radio-frequency. In addition, short-range communication may occur, such as using a Bluetooth, Wi-Fi, or other such transceiver (not shown). In addition, a GPS (Global Positioning System) receiver module 770 may provide additional navigation- and location-related wireless data to the mobile computing device 750, which may be used as appropriate by applications running on the mobile computing device 750.

The mobile computing device 750 may also communicate audibly using an audio codec 760, which may receive spoken information from a user and convert it to usable digital information. The audio codec 760 may likewise generate audible sound for a user, such as through a speaker, e.g., in a handset of the mobile computing device 750. Such sound may include sound from voice telephone calls, may include recorded sound (e.g., voice messages, music files, etc.) and may also include sound generated by applications operating on the mobile computing device 750.

The mobile computing device 750 may be implemented in a number of different forms, as shown in the figure. For example, it may be implemented as a cellular telephone 780. It may also be implemented as part of a smart-phone 782, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., an OLED (organic light emitting diode) display or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some embodiments, the computing system can be cloud based and/or centrally processing data. In such case anonymous input and output data can be stored for further analysis. In a cloud based and/or processing center set-up, compared to distributed processing, it can be easier to ensure data quality, and accomplish maintenance and updates to the calculation engine, compliance to data privacy regulations and/or troubleshooting.

A number of implementations have been described. Other implementations are in the following claims.

What is claimed is:

1. A computer-implemented biological data classification method executed by one or more processors and comprising:
receiving, by the one or more processors, a first biological data set comprising a first plurality of biological sample data collected from a set of patients;
processing, by the one or more processors, the first biological data set using a first variational autoencoder (VAE) to generate a first trained VAE comprising a first latent space vector of the first biological data set comprising a plurality of values corresponding to each latent space dimension of the latent space vector, the latent space vector having lower dimensionality than the biological sample data set;
receiving, by the one or more processors, a second biological data set comprising a second plurality of biological sample data collected from a patient, different from the set of patients; and
generating, by the one or more processors, a latent space representation of the second biological data set based on a first latent space vector.

2. The method of claim 1, wherein the first VAE is a βVAE.

3. The method of claim 1, wherein the first biological data set comprises analyte volumes in collected biological samples.

4. The method of claim 3, wherein the collected biological samples are blood, serum, saliva, plasma, interstitial fluid.

5. The method of claim 3, wherein the analyte volumes comprise protein volumes, or metabolite volumes

6. The method of claim 1, further comprising corrupting the received data set using a corruption function.

7. The method of claim 6, wherein prior to receiving, the method comprises corrupting or ablating the first biological data set.

8. The method of claim 6, wherein the corruption function is a salt and pepper function, a Gaussian function, or a masking function.

9. The method of claim 1, wherein a loss function of the first VAE is a forward KL divergence model, or a reverse KL divergence model.

10. The method of claim 1, further comprising:
receiving, by the one or more processors, a classification label data set comprising a plurality of classification label constructed from biological sample data labels;
processing, by the one or more processors, the classification label data set using a second VAE to generate a second trained VAE comprising a second latent space vector of the classification label data set, the second latent space vector comprising a plurality of values corresponding to each latent space dimension of the classification label data, the latent representation having lower dimensionality than the classification label data set;
communicating, by the one or more processors, the latent space representation of the second biological data set to the second VAE; and
classifying, by the one or more processors, the latent space representation of the second biological data set based on a first latent space vector.

11. The method of claim 10, wherein the classifying comprises generating a disease prediction based on the second biological data set.

12. The method of claim 10, the method further comprising:
receiving, by the one or more processors, a set of classifications comprising a plurality of sample classifications;
processing, by the one or more processors, the set of classifications using the second VAE to generate a classification representation of the set of classifications, the second latent representation having lower dimensionality than the set of classifications;
communicating, by the one or more processors, the classification representation to the first VAE; and
generating, by the one or more processors, a predicted biological data set based on the classification representation.

13. A system comprising:
at least one processor; and
a storage device coupled to the at least one processor having instructions stored thereon which, when executed by the at least one processor, causes the at least one processor to perform operations comprising:
receiving, by the one or more processors, a first biological data set comprising a first plurality of biological sample data collected from a set of patients;
processing, by the one or more processors, the first biological data set using a first variational autoencoder (VAE) to generate a first trained VAE comprising a first latent space vector of the first biological data set comprising a plurality of values corresponding to each latent space dimension of the latent space vector, the latent space vector having lower dimensionality than the biological sample data set;
receiving, by the one or more processors, a second biological data set comprising a second plurality of biological sample data collected from a patient, different from the set of patients; and
generating, by the one or more processors, a latent space representation of the second biological data set based on a first latent space vector.

14. The system of claim 13, the data store further having further instructions stored thereon including a second VAE.

15. The system of claim 14, the data store further having further instructions stored thereon which, when executed by the at least one processor, causes the at least one processor to perform operations comprising:
receiving, by the one or more processors, a classification label data set comprising a plurality of classification label constructed from biological sample data labels;
processing, by the one or more processors, the classification label data set using the second VAE to generate a second trained VAE comprising a second latent space vector of the classification label data set, the second latent space vector comprising a plurality of values corresponding to each latent space dimension of the classification label data, the latent representation having lower dimensionality than the classification label data set;
communicating, by the one or more processors, the latent space representation of the second biological data set to the second VAE; and
classifying, by the one or more processors, the latent space representation of the second biological data set based on a first latent space vector.

16. The system of claim 14, the data store further having further instructions stored thereon which, when executed by the at least one processor, causes the at least one processor to perform operations comprising:
receiving, by the one or more processors, a set of classifications comprising a plurality of sample classifications;
processing, by the one or more processors, the set of classifications using the second VAE to generate a classification representation of the set of classifications, the second latent representation having lower dimensionality than the set of classifications;
communicating, by the one or more processors, the classification representation to the first VAE;
generating, by the one or more processors, a predicted biological data set based on the classification representation.

17. A non-transitory computer readable storage medium storing instructions that, when executed by at least one processor, cause the at least one processor to perform operations comprising:
receiving, by the one or more processors, a first biological data set comprising a first plurality of biological sample data collected from a set of patients;
processing, by the one or more processors, the first biological data set using a first variational autoencoder (VAE) to generate a first trained VAE comprising a first latent space vector of the first biological data set comprising a plurality of values corresponding to each latent space dimension of the latent space vector, the latent space vector having lower dimensionality than the biological sample data set;
receiving, by the one or more processors, a second biological data set comprising a second plurality of biological sample data collected from a patient, different from the set of patients; and
generating, by the one or more processors, a latent space representation of the second biological data set based on a first latent space vector.
